(19) United States
(12) Patent Application Publication
(10) Pub. No.: US 2008/0050776 A1
(43) Pub. Date: Feb. 28, 2008

(54) Stable mutated pro nerve growth factors

(76) Inventors: Kenneth Neet, Deerfield, IL (US); Promila Pagadala, North Chicago, IL (US)

Correspondence Address: Joseph A. Fuchs;Rockey, Depke & Lyons, LLC, 233 S. Wacker Drive, Suite 5450, Sears Tower, Chicago, IL, 60606-6306, US

(21) Appl. No.: 11/807,056

(22) Filed: May 25, 2007

Related U.S. Application Data
(60) Provisional application No. 60/808,919, filed on May 26, 2006.

(51) Int. Cl. C12P 21/04; C07H 21/04; C07K 14/00

(52) U.S. Cl. 435/69.1; 530/350; 536/23.5

(57) ABSTRACT

The present invention relates generally to novel stable pro nerve growth factors (proNGFs) that are stable towards proteolysis by proteases such as furin, PACE4, and PC2. The novel proNGF molecules are prepared with multiple mutations at all the three major processing sites of the pro domain of the natural proNGF molecules. The present invention further discloses the construction, stable expression in insect cells, and purification of stable mutated proNGF molecules. These novel stable proNGF molecules are useful as a reagent to study the physiological process of apoptosis of neuronal cells. Clinically, the stable proNGF molecules, or further mutants derived from them, have potential use in treating certain cancers such as neuroblastoma, pancreatic and breast cancer as well as used as a target in developing other therapeutic agents for neurodegenerative disorders.

### CROSS-REFERENCE TO RELATED APPLICATION

This application claims priority to U.S. Provisional patent application Ser. No. 60/808,919 filed on May 26, 2006, which is incorporated herein by reference in its entirety and made a part hereof.

### FEDERALLY SPONSORED RESEARCH OR DEVELOPMENT

This invention was made with government support under National Institutes of Health grant number NS24380. The government has certain rights in the invention.

### BACKGROUND OF THE INVENTION

1. Field of the Invention

The present invention relates generally to novel stable pro nerve growth factors (proNGFs) that are stable towards proteolysis by proteases such as furin, PACE4, and PC2. The novel proNGF molecules are prepared with multiple mutations at all the three major processing sites of the pro domain of the natural proNGF molecules. The present invention further discloses the construction, stable expression in insect cells, and purification of stable mutated proNGF molecules. These novel stable proNGF molecules are useful as a reagent to study the physiological process of apoptosis of neuronal cells. Clinically, the stable proNGF molecules, or further mutants derived from them, have potential use in treating certain cancers such as neuroblastoma, pancreatic and breast cancer as well as used as a target in developing other therapeutic agents for neurodegenerative disorders.

2. Background of the Invention

The neurotrophin family includes structurally related proteins that promote the survival, growth and maintenance of neurons in the central and peripheral nervous system (Bibel, M & Barde, Y A (2000) Neurotrophins: key regulators of cell fate and cell shape in the vertebrate nervous system. Genes Dev 14, 2919-37). Nerve growth factor (NGF), the first member of the family, was discovered by Levi-Montalcini and coworkers over 50 years ago (Levi-Montalcini, R (1987) The nerve growth factor 35 years later. Science 237, 1154-1162). Other members of the family include: brain-derived neurotrophic factor (BDNF), neurotrophin-3 (NT-3), NT-4/5 and NT-6 (3,13,15,16). Neurotrophins play a crucial role in neuronal survival, differentiation, growth, and apoptosis (Barbacid, M (1994) The Trk family of neurotrophin receptors. J. Neurobiol. 25, 1386-1403). Each neurotrophin binds to a 140 kDa tyrosine kinase receptor, known as Trk receptor. NGF binds to TrkA, BDNF and NT4/5 selectively bind to TrkB, and NT-3 to TrkC (Bibel, M & Barde, Y A (2000) Neurotrophins: key regulators of cell fate and cell shape in the vertebrate nervous system. Genes Dev 14, 2919-37; Bibel, M, Hoppe, E & Barde, Y A (1999) Biochemical and functional interactions between the neurotrophin receptors trk and p75NTR. EMBO J 18, 616-22). In addition to a selective Trk receptor for each neurotrophin, there is a common neurotrophin receptor-p75 NTR. The two receptors, Trk and p75, are structurally unrelated with neurotrophins interacting with the immunoglobulin-like C2 (IgGC2) domains of the Trk receptors but with the cysteine-rich domains of the p75NTR receptor (Baldwin, A N, Bitler, C M, Welcher, A A & Shooter, E M (1992) Studies on the structure and binding properties of the cysteine-rich domain of rat low affinity nerve growth factor receptor (p75NGFR). J. Biol. Chem. 267, 8352-8359; Wiesmann, C & de Vos, A M (2001) Nerve growth factor: structure and function. Cell Mol Life Sci 58, 748-59; Wiesmann, C, Ultsch, M H, Bass, S H & de Vos, A M (1999) Crystal structure of nerve growth factor in complex with the ligand-binding domain of the TrkA receptor. Nature 401, 184-188). By binding to Trk receptors, the neurotrophins can signal growth, survival, or differentiation, i.e. the positive signals, while binding to heterodimer can transduce positive or negative signals (Eide, F F, Lowenstein, D H & Reichardt, L F (1993) Neurotrophins and their receptors—current concepts and implications for neurologic disease. Exp. Neurol. 121, 200-214; Friedman, W J (2000) Neurotrophins induce death of hippocampal neurons via the p75 receptor. J Neurosci 20, 6340-6; Friedman, W J & Greene, L A (1999) Neurotrophin signaling via Trks and p75. Exp Cell Res 253, 131-42; Mufson, E J, Lavine, N, Jaffar, S, Kordower, J H, Quirion, R & Saragovi, H U (1997) Reduction in p140-TrkA receptor protein within the nucleus basalis and cortex in Alzheimer's disease. Exp Neurol 146, 91-103). Unlike full-length Trk receptors that possess signature tyrosine kinase motifs, p75NTR lacks any intrinsic catalytic activity. It mediates signals through a series or assembly of adaptor proteins (Bibel, M & Barde, Y A (2000) Neurotrophins: key regulators of cell fate and cell shape in the vertebrate nervous system. Genes Dev 14, 2919-37; Bibel, M, Hoppe, E & Barde, Y A (1999) Biochemical and functional interactions between the neurotrophin receptors trk and p75NTR. EMBO J 18, 616-22; Kong, H, Kim, A H, Orlinick, J R & Chao, M V (1999) A comparison of the cytoplasmic domains of the Fas receptor and the p75 neurotrophin receptor. Cell Death Differ. 6, 1133-42). The role of p75 in cell signaling is being found to be more and more important.

Mature NGF is a 118-120 amino acid protein. The subunit is a 13 kDa polypeptide with three disulfide bonds forming a cystine knot (McInnes, C & Sykes, B D (1997) Growth factor receptors: structure, mechanism, and drug discovery. Biopolymers. 43, 339-66). NGF exists as a 26.5 kDa non covalent dimer with the subunits arranged in parallel orientation (Maness, L M, Kastin, A J, Weber, J T, Banks, W A, Beckman, B S & Zadina, J E (1994) The neurotrophins and their receptors: structure, function, and neuropathology. Neurosci. Biobehav. Rev. 18, 143-159). Like other growth factors, NGF is synthesized as an immature precursor, proNGF (Server, A C & Shooter, E M (1977) Nerve growth factor. Adv. Protein. Chem. 31, 339-409). Because of homology in the pro parts of the neurotrophins, the precursor was thought to have a role in proper folding, sorting to either constitutive or regulated pathway, and secretion of the mature neurotrophin (Rattenholl, A, Lilie, H, Grossmann, A, Stern, A, Schwarz, E & Rudolph, R (2001) The pro-sequence facilitates folding of human nerve growth factor from Escherichia coli inclusion bodies. Eur J Biochem 268, 3296-303; Seidah, N G, Benjannet, S, Pareek, S, Savaria, D, Hamelin, J, Goulet, B, Laliberte, J, Lazure, C, Chretien, M & Murphy, R A (1996) Cellular processing of the nerve growth factor precursor by the mammalian pro-protein convertases. Biochem J 314, 951-60). Contrary to the earlier beliefs, proNGF has now been found to be the high affinity ligand for the receptor p75 and also the molecule responsible for inducing apoptosis mediated by p75 more effectively than mature NGF in some systems (Harrington, A W, Leiner, B, Blechschmitt, C, Arevalo, J C, Lee, R, Morl, K, Meyer, M, Hempstead, B L, Yoon, S O & Giehl, K M (2004) Secreted proNGF is a pathophysiological death-inducing ligand after adult CNS injury. Proc Natl Acad Sci USA 101, 6226-30. Epub Mar. 16, 2004; Lee, R, Kermani, P, Teng, K K & Hempstead, B L (2001) Regulation of cell survival by secreted proneurotrophins. Science 294, 1945-8; Nykjaer, A, Lee, R, Teng, K K, Jansen, P, Madsen, P, Nielsen, M S, Jacobsen, C, Kliemannel, M, Schwarz, E, Willnow, T E, Hempstead, B L & Petersen, C M (2004) Sortilin is essential for proNGF-induced neuronal cell death. Nature 427, 843-8). Following translation of NGF mRNA, post-translational glycosylations, sulfation, and limited proteolytic cleavage at conserved basic sites gives rise to the mature NGF. The precursor of NGF, pre-pro-NGF, is 31-35 kDa in size and has its N-terminus hydrophobic signal peptide followed by pro region and mature region. ProNGF is 241 amino acids in length and has a molecular weight of about 32 kDa. The pro region contains two sites for N-glycosylations and three separate sequences of two or more contiguous basic amino acids (Kliemannel, M, Rattenholl, A, Golbik, R, Balbach, J, Lilie, H, Rudolph, R & Schwarz, E (2004) The mature part of proNGF induces the structure of its pro-peptide. FEBS Lett 566, 207-12; Rattenholl, A, Lilie, H, Grossmann, A, Stern, A, Schwarz, E & Rudolph, R (2001) The pro-sequence facilitates folding of human nerve growth factor from Escherichia coli inclusion bodies. Eur J Biochem 268, 3296-303; Seidah, N G, Benjannet, S, Pareek, S, Savaria, D, Hamelin, J, Goulet, B, Laliberte, J, Lazure, C, Chretien, M & Murphy, R A (1996) Cellular processing of the nerve growth factor precursor by the mammalian pro-protein convertases. Biochem J 314, 951-60). Intracellular cleavage of the proNGF to produce active NGF takes place following pairs of basic amino acids of the type I precursor motif Arg-Xaa-Lys/Arg-ArgX, where Xaa is Ser, Val, and Arg for proNGF. ProNGF is processed into mature NGF following the arrival of the precursor in the trans-Golgi network (Seidah, N G, Benjannet, S, Pareek, S, Savaria, D, Hamelin, J, Goulet, B, Laliberte, J, Lazure, C, Chretien, M & Murphy, R A (1996) Cellular processing of the nerve growth factor precursor by the mammalian pro-protein convertases. Biochem J 314, 951-60). It was earlier shown that the major cleavage site for processing of precursor to mature NGF is located at −1 and −2 amino acid positions (termed site 3) of the NGF cDNA (Heymach Jr., J V, Kruttgen, A, Suter, U, Shooter, E M (1996) The Regulated Secretion and Vectorial Targeting of Neurotrophins in Neuroendocrine and Epithelial Cells. JBC 271, 25430-25437). Most of the recent research on proNGF has been done on proNGF mutated at this major site. Our experiments show that site 3 is not the only site involved in processing of proNGF. Mutating site 3 renders proNGF only partially stable. The present invention discloses that mutation of each of the other two possible processing sites (termed sites 1 and 2) significantly enhances the stability of proNGF and thus renders it much more suitable for biophysical, cellular, and animal studies, as well as a enhancing the potential as a therapeutic agent.

These and other aspects and attributes of the present invention will be discussed with reference to the following drawings and accompanying specification.

### SUMMARY OF THE INVENTION

The present invention relates generally to novel stable pro nerve growth factors (proNGFs) that are stable towards proteolysis by proteases such as furin, PACE4, and PC2. The novel proNGF molecules are prepared with multiple mutations at all the three major processing sites of the pro domain of the natural proNGF molecules. The present invention further discloses the construction, stable expression in insect cells, and purification of stable mutated proNGF molecules. These novel stable proNGF molecules are useful as a reagent to study the physiological process of apoptosis of neuronal cells. Clinically, the stable proNGF molecules, or further mutants derived from them, have potential use in treating certain cancers such as neuroblastoma, pancreatic and breast cancer as well as used as a target in developing other therapeutic agents for neurodegenerative disorders.

In one embodiment, the present invention relates to a mutated mammalian stable pro nerve growth factor (proNGF) derived from a wild type mammalian proNGF, the wild type proNGF having a pro domain and a mature domain, a first dibasic protease sensitive site with contiguous basic amino acids in the pro domain, a second dibasic protease sensitive site with contiguous basic amino acids in the pro domain, and a third dibasic protease sensitive site with contiguous basic amino acids in the pro domain, wherein each of the basic amino acid residue in each of the dibasic site in the wild type proNGF is replaced by a non-basic amino acid and wherein the mutated proNGF is resistant to cleavage by proteases to form a mature NGF having only the mature domain of the wild type proNGF. The non-basic amino acid is preferably a neutral amino acid, and more preferably, alanine. The stable mutated proNGF is preferably a human proNGF.

In yet another embodiment, the invention relates to a method for making a mutated stable mammalian pro nerve growth factor (proNGF) from a wild type proNGF, the wild type proNGF having a pro domain and a mature domain, a first dibasic protease sensitive site having contiguous amino acids in the pro domain, a second dibasic protease sensitive site having contiguous amino acids in the pro domain, and a third dibasic protease sensitive site having contiguous amino acids in the pro domain, the method comprising: (a) providing a cDNA for the wild type proNGF; (b) subcloning the cDNA into a cell vector such as the baculovirus; (c) mutating the cDNA at sites corresponding to each of the dibasic sites to non-basic amino acids to obtain a plasmid with mutated cDNA in the cell vector; (d) transfecting the plasmid or the cell vector containing the plasmid into a host cell; and (e) culturing the host cell to allow expression of the mutated proNGF; wherein the mutated proNGF is resistant to cleavage by proteases to form a mature NGF having only the mature domain of the wild type proNGF. Preferably, a native stop codon in the cDNA is induced to avoid the 6-histidine residue (6 His) tag in the vector. In another preferred embodiment, the mutated stable proNGF is further purified from the cell culture. In yet another preferred embodiment, the cell transfected by the plasmid is an insect cell, preferably a Sf-21, Sf-9, or Hi-5 insect cell.

### BRIEF DESCRIPTION OF THE DRAWINGS

FIG. 1 is a schematic diagram of a mammalian proNGF molecule showing the three major processing sites, Site 1, Site 2, and Site 3;

FIGS. 2A and B are the results of the Western Blots showing (A) processing of proNGF modified at sites 1 and 2, (B) processing of proNGF modified at site 2;

FIGS. 3A and B are the results of the Western Blots showing the processing of proNGF-3. Lane 1 of FIG. 3A is 15 ng mouse βNGF, Lane 2 of FIG. 3A shows the elution fractions for proNGF(R-1>A). Lane 1 of FIG. 3B is 15 ng mouse βNGF and Lane 2 of FIG. 3B shows the expression of proNGF (KR>AA);

FIGS. 4A and B are the results of the Western Blots showing the processing of proNGF-3,1 (FIG. 4A) and proNGF-3,2 (FIG. 4B);

FIGS. 5A and B show the results of the expression and purification of proNGF-1,2,3. FIG. 5A is a Western Blot showing complete resistance of proNGF-1,2,3 to processing enzymes and FIG. 5B shows the result of the purification of proNGF-1,2,3;

FIGS. 6A and B show the post-translational modifications of proNGF-1,2,3. FIG. 6A is a Coomassie staining of proNGF-1,2,3, and FIG. 6B is a glycoprotein staining of the same;

FIGS. 7A, B and C show the results of the neurite growth assay. FIG. 7A is the result of PC-12 cells treated with 2 nM of mature NGF showing neurites in 72 hours in complete DMEM medium. FIG. 7B is the result of PC-12 cells treated with 2 nM of proNGF-1,2,3 showing no neurites after 168 hours. FIG. 7C is the result of PC-12 cells treated with buffer showing no neurites;

FIGS. 7D, E, and F show the results of cell live/dead survival assays. FIG. 7D shows PC-12nnr cells treated with 2 nM mature NGF for 72 hours are alive in complete DMEM medium. FIG. 7E shows PC-12nnr cells treated with 2 nM proNGF-1,2,3 show more than 60% cell death within 72 hours. FIG. 7F shows PC-12nnr cells treated with buffer do not show significant cell death;

FIG. 8 shows the quantification of cell survival using the XTT assay after treatment with recombinant NGF (open bars) or proNGF123 (filled bars) at the concentrations indicated for 72 hours in defined DMEM. A) RN22 schwannoma cells, B) C6 glioma cells, and C) PC12nnr cells show similar survival as negative control on being treated with various concentrations of NGF while higher cell death is observed with increasing concentration of proNGF 123.

FIG. 9A is a Western Blot showing the resistance of proNGF123 to processing enzymes; FIG. 9B is a Western Blot showing the susceptibility of proNGF3 to the same processing enzymes.

FIG. 10 is an amino acid sequence (SEQ ID NO:7) of the mutated mouse proNGF123 translated from the mutated cDNA shown in FIG. 11 (SEQ ID NO:8);

FIG. 11 is a cDNA sequence (SEQ ID NO:8) for the amino acid sequence (SEQ ID NO:7) of the mutated mouse proNGF123;

FIG. 12 is a comparison of the amino acid sequence of mouse proNGF (SEQ ID NO:9) and the amino acid sequence of human proNGF (SEQ ID NO:10);

FIG. 13 is an amino acid sequence (SEQ ID NO:11) of the mutated human proNGF123 with all the dibasic amino acids in the protease sensitive sites each replaced by alanine.

### DETAILED DESCRIPTION OF THE INVENTION

While this invention is susceptible of embodiments in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

ProNGF has been found to be pro-apoptopic in several cell types. It mediates its effects by binding to p75 neurotrophin receptor (NTR) and the co-receptor sortilin to promote cell death in neurons possessing these receptors.

An existing challenge for structural and cellular studies with proNGF is the production of sufficient quantity of stable and biologically active protein. A stable proNGF is needed to study the physiological process of apoptosis of neuronal cells. Clinically, a stable proNGF, or further mutants derived from it, has potential use in certain cancers such as neuroblastoma, pancreatic or breast cancer, as well as a target for developing other therapeutic agents to treat neurodegenerative diseases.

The present invention discloses novel stable pro nerve growth factors (proNGFs) that are stable towards proteolysis by proteases such as furin, PACE4, and PC2. The novel proNGF molecules are prepared with multiple mutations at all three major protease sensitive dibasic sites of the pro domain of the natural proNGF. The present invention further discloses the construction, stable expression in insect cells, and purification of mutated stable proNGF molecules.

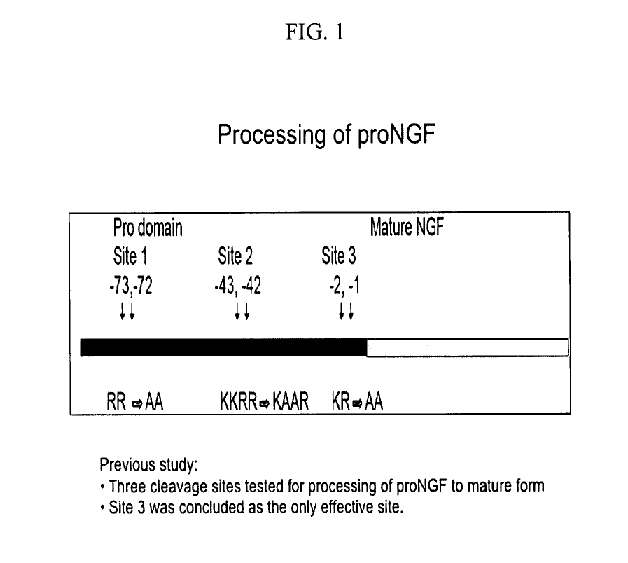

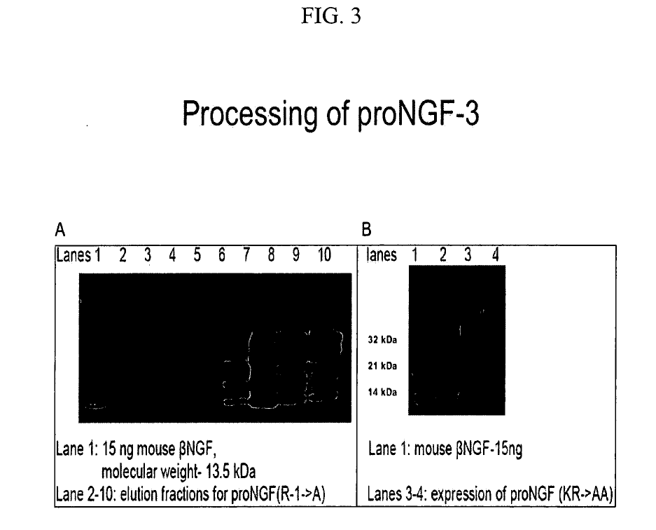

There are three major protease sensitive dibasic sites, also known as the processing sites, in the pro domain of proNGF (FIG. 1). Each of the dibasic sites has contiguous basic amino acids. The proNGF molecule is processed by proteases at these dibasic sites to form mature NGF. These protease sensitive sites are named site 1, site 2 and site 3 from the N-terminus. As shown in FIG. 1, site 1 is located at −73 and −72 amino acid positions of the NGF molecule, site 2 at the −43 and −42 amino acid positions, and site 3 at the −1 and −2 amino acid positions. In an earlier study, site 3 was shown to be the “major” processing site while sites 1 and 2 did not show any effect on processing of proNGF to NGF on their own (Heymach Jr., J V, Kruttgen, A, Suter, U, Shooter, E M (1996) The Regulated Secretion and Vectorial Targeting of Neurotrophins in Neuroendocrine and Epithelial Cells. JBC 271, 25430-25437). Although site 3 is a major site, mutating site 3 to form proNGF-3 does not make proNGF completely stable (see Example 5 below, FIG. 3).

In the present disclosure, the novel stable proNGF molecules are produced by mutating each of the three protease sensitive dibasic sites by mutating the wild type cDNA for proNGF at sites corresponding to each of the basic amino acid at the dibasic sites to non-basic residues. The non-basic amino acid residue can be acidic or neutral. A preferred neutral amino acid is alanine. The product is a noncleaved and stable proNGF protein that is resistant to proteases and processing enzymes.

United State Patent Application No. US2003/0087804A1 by Hempstead et al. discloses “cleavage resistant” proNGF molecules prepared by replacing a single “major” proNGF cleavage site (site 3) with a connector. The connector is a chemical bond or any chemical group, such as an amino acid sequence, that is capable of stably joining the pro domian and the mature domain. The resultant molecules are “cleavage resistant” to certain proteinases. However, this patent application does not provide any stability data for these molecules. Our studies show that mutation or modification at only this major site results in molecules that are only partially stable (see Example 5 below, FIG. 3).

The novel proNGF molecule in the present invention, known as proNGF-1,2,3 or proNGF123 herein, is derived from a proNGF from any mammalian species, preferably a human proNGF. The proNGF can be glycosylated or unglycosylated. In a preferred embodiment, the proNGF is glycosylated. In the present disclosure, proNGF is referred to the precursor of NGF. The proNGF molecule has a pro domain and a mature NGF domain. Upon processed by a processing enzyme, the proNGF gives rise to the mature NGF molecule with only the mature NGF domain. The NGF molecule includes any natural NGF molecules, which are well known as well defined in the scientific literature, as well as NGF molecules derived therefrom that have similar biological activities as the natural NGF molecules by binding to the TrkA receptor.

In a preferred embodiment, the novel stable proNGF is prepared by mutating the wild type cDNA for proNGF at sites corresponding to each of the dibasic sites described earlier (site 1, site 2 and site 3) to non-basic residues by replacing each of the six basic amino acids with a non-basic amino acid. The non-basic amino acid used to replace all the six basic amino acids at the three dibasic sites can be the same, or they can be different and can be in any combination. Any non-basic amino acid can be used to replace the basic amino acid residues, including both acidic amino acids or neutral amino acids. In a preferred embodiment, the non-basic amino acid is a neutral amino acid. In another preferred embodiment, the neutral amino acid is alanine. In yet another embodiment, all the six basic amino acids in the three dibasic sites are replaced with the neutral amino acid alanine. Mutation is accomplished, for example, by first subcloning a proNGF cDNA into a cell vector, such as a baculovirus. In a preferred embodiment, a native stop codon in the cDNA is induced to avoid the 6 His residue tag in the vector. Sequential site directed mutagenesis is performed with separate primers for each of the three site mutation. Plasmids with mutated cDNA are amplified, for example, in E. coli, and purified for transfection grade DNA. An appropriate host cell is transfected with the mutated proNGF cDNA plasmid or the plasmid incorporated in the cell vector. Appropriate host cells suitable for transfection are well known to those skilled in the art. Examples of appropriate host cell for transfection include but are not limited to mammalian cells (e.g. Chinese Hamster Ovarian, or CHO, cells), avian cells, insect cells, plant cells, yeast cells and the like. In an embodiment, an insect cell is used as the host cell for the transfection. In yet another embodiment, Sf-21, Sf-9, or Hi-5 is used for the as the host cell for the transfection and the cell vector is the pIZT/his/V5 vector. The transfected cell is grown in an appropriate cell culture medium to allow the cell to express the mutated proNGF protein, which can be isolated and purified from the expression medium using one or more bio-separation and purification techniques. Such techniques are well known to those skilled in the art, and include, but are not limited to, centrifugation, chromatography, electrophoresis, isoelectrofocusing, dialysis and the like.

The resulting mutated proNGF (proNGF123) molecules are resistant to cleavage by proteases. What is meant by “resistant to cleavage by proteases” in the present invention is that the mutated proNGF molecules have longer half-lives as compared to the non-mutated, wild-type proNGF when subjected to cleavage by proteases such as furin, PACE4, and PC2, which are commonly known to rapidly cleave wild type proNGF molecules. However, it is not necessary that the mutated proNGF is resistant to cleavage by all proteases. Like the wild type proNGF, these proNGF123 molecules are biologically active in inducing apoptosis. While mature NGF induces neurite outgrowth and stimulates survival in PC-12 cells which express TrkA receptor, p75NTR and the Sortilin co-receptor, proNGF123 induces cell death in these PC-12 cells.

Alternatively, the stable novel mutated proNGF123 molecules can be produced by any other peptide or protein synthetic or biosynthetic methods known in the art, such as using the peptide/protein synthesizer.

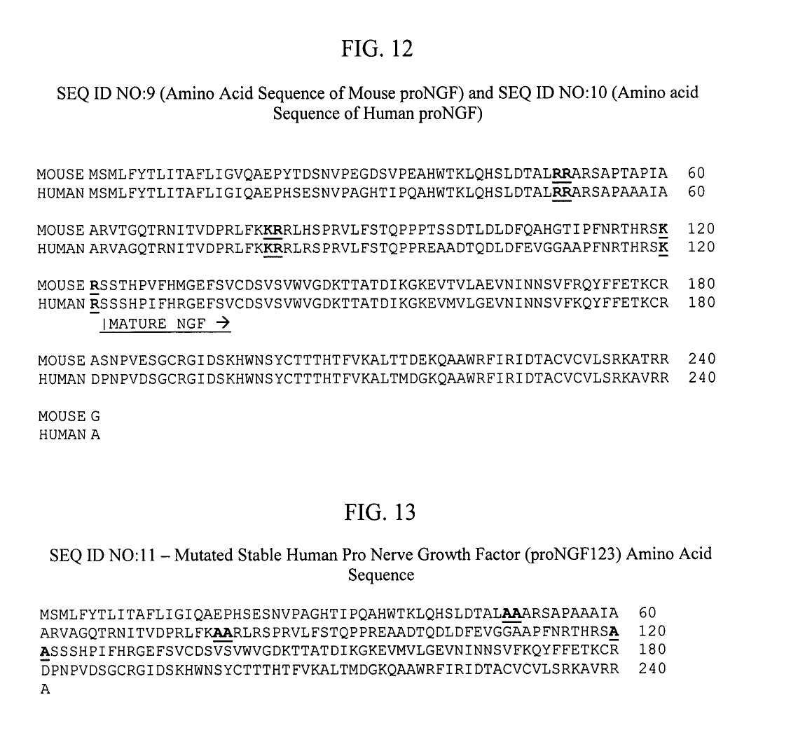

The present disclosure uses the mouse proNGF for the mutation to form the novel stable proNGF123, which exemplifies the invention disclosed herein. It is anticipated that other mammalian proNGFs, particularly human proNGF, can be similarly modified to derive stable proNGFs due to the high homologies of the amino acid sequences of the pro domains of the mammalian proNGF's. In addition, mouse and human proNGFs share the same dibasic protease sensitive sites. FIG. 12 provides a comparison of the amino acid sequence of the mouse proNGF (SEQ ID NO:9) and the amino acid sequence of the human proNGF (SEQ ID NO:10) with the dibasic sites bolded and underlined. The amino acid sequence of the mutated human proNGF123 with all the basic amino acids each replaced by alanine is shown in FIG. 13 (SEQ ID NO:11).

These novel stable proNGF123 molecules are useful as a reagent to study the physiological process of apoptosis of neuronal cells. Clinically, the stable proNGF123 molecules, or further mutants derived from them, have potential use in treating certain cancers such as neuroblastoma, pancreatic and breast cancer as well as used as a target in developing other therapeutic agents for neurodegenerative disorders.

### EXAMPLES

### Example 1

### Cell Cultures

PC-12 and PCnnr cells were grown in Dulbecco's Modified Eagle Media (DMEM) (cellgro) supplemented with 10% Horse serum, 5% FBS, 4.5 mg/ml glucose, 4.0 mM L-glutamine, 100 units/ml penicillin, 100 pg/ml streptomycin, and 0.25 pg/ml amphotericin-B25 at 37° C. in a humid atmosphere containing 5% CO₂. They were subcultured every 72 hours at a ratio of 1:3. Treatments of PC-12 and PCnnr cells were carried out in the same medium as subculturing immediately after plating in collagen coated 96 well plates.

Sf-21 insect cells were grown in Graces insect cell medium (GIBCO, Langley, Okla.) supplemented with 10% FBS at 27° C. they were subcultured every 72 hours at a ratio of 1:4. Stably transfected Sf-21 insect cells expressing proNGF mutants were grown in Grace's insect cell medium supplemented with 10% FBS and 400 μg/ml zeocin. Transfected Sf-21 cells were grown at 27° C. and subcultured every 48 hours at a ratio of 1:2.

### Example 2

### Plasmid Constructs and Production of Stable Transfectants

Mouse β NGF cDNA was subcloned in insect cell vector pIZT/his/V5 (Invitrogen, Carlsbad, Calif.) at the KpnI-AgeI restriction sites of the polylinker region. Native stop codon in the cDNA of mouse β NGF was included to avoid the 6 His residue tag in the vector. Sequential site directed mutagenesis was performed with separate primers for each site mutations.

For site 1, forward primer (SEQ ID NO:1):

CCTTGACACAGCCCTCGCCGCAGCCCGCAGTGCCCCTACTGCACCAATAG

Site 1, reverse primer (SEQ ID NO:2):

GGAACTGTGTCGGGAGCGGCGTCGGGCGTCACGGGGATGACGTGGTTATC

For site 2, forward primer (SEQ ID NO.3):

CCCAGACTGTTTAAGGCAGCGAGACTCCACTCACCCCGTGTGCTGTTCAG

Site 2, reverse primer (SEQ ID NO:4):

GGGTCTGACAAATTCCGTCGCTCTGAGGTGAGTGGGGCACACGACAAGTC

For site 3, forward primer (SEQ ID NO:5):

CAGGACTCACCGGAGCGCGGCCTCATCCACCCACCCAGTCTTCCACATGG

Site 3, reverse primer (SEQ ID NO:6)

GTCCTGAGTGGCCTCGCGCCGGAGTAGGTGGGTGGGTCAGAAGGTGTACC

All constructs were analyzed by sequence analyses to verify the mutations and the correct frame of reading. Plasmids with mutated cDNAs were amplified in E. coli and purified using QIAGEN (Valencia, Calif.) maxi prep kit for transfection grade DNA.

Sf-21 insect cells were transformed with the mutated proNGF cDNA incorporated into the vector pIZT using lipofectamine reagent (Invitrogen, Carlsbad, Calif.) according to the manufacturer's instructions. Transfected cells were selected using 500 μg/ml zeocin in the medium. Zeocin concentration to be used for selecting the stable transfectants was calculated by performing a kill curve on non transfected insect cells.

Although mouse β NGF cDNA is used in this example, those skilled in the art can readily replace mouse β NGF with any other mammalian proNGF cDNAs, such as the human proNGF cDNA to make a stable human proNGF. Alternatively, proNGF can be replaced by other mammalian neurotrophins, such as BDNF, NT-3, or NT-4/5.

### Example 3

### Expression and Purification of Mutated proNGF

For expression of mutated proNGF, stably transfected insect cells were grown in spinners in Excel-401 medium with 100 μg/ml zeocin added every 72 hours for about 8 days. After 8 days, expression medium was centrifuged for clarification and loaded onto SP fast flow column (Amersham-GE, Piscataway, N.J.) for strong cation exchange chromatography. Two washes were given with 20 mM tris pH 8.0 and 50 mM tris pH 9.0. The proNGF was eluted with 400 mM NaCl, 50 mM tris, pH 9.0. Fractions containing proNGF were then loaded onto N-60 immunoaffinity column, washed with 400 mM NaCl, 50 mM tris, pH 9.0, and 20 mM tris, pH 8.0. ProNGF was eluted with 0.1 N glycine pH 2.6 and the pH was restored to 7.0 with 2 M tris. The degree of purification of proNGF after these two columns is about 300 fold.

### Example 4

### Cellular Assays

Neurite outgrowth and live cell assays were performed with PC-12 cells and PCnnr cells on collagen coated 96 well plates. PC-12 and PCnnr cells were resuspended in complete DMEM and diluted to approximately 30,000 cells/ml and 100 μl of cell suspension was added to each well of collagen coated 96 well plate. PC-12 and PCnnr cells were then treated with mNGF, proNGF, and buffer for 6 days in complete DMEM. Fresh media were replaced every 48 hours. Between day 3 and day 6, neurites were counted at regular intervals and at the end of six days a live cell assay was performed with trypan blue dye.

### Example 5

### Effects of Mutation at the Three Protease Sensitive Sites of proNGF

Although site 3 is a major site, mutating site 3 does not make proNGF completely stable (see FIG. 3). Since the proNGF mutated at site 3 is only partially stable, additional mutations were made to this proNGF construct. ProNGF mutants were made with various combinations of sites mutated—sites 3 and 1, sites 3 and 2, sites 1 and 2, and sites 1, 2, and 3.

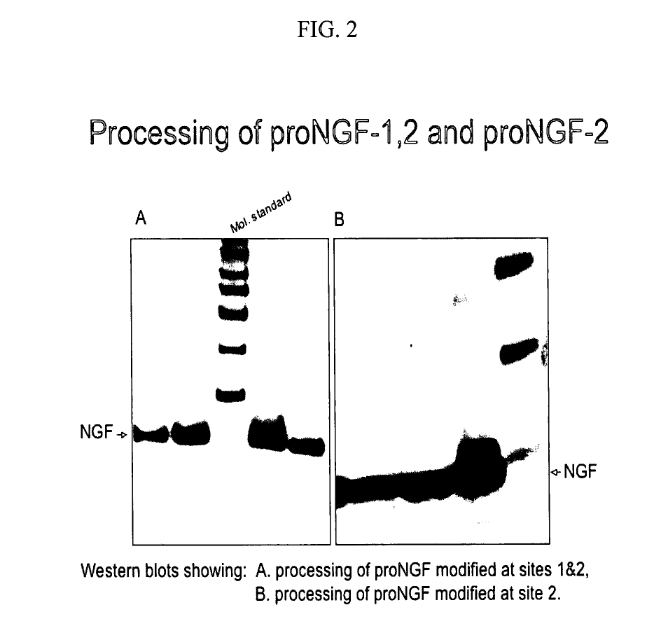

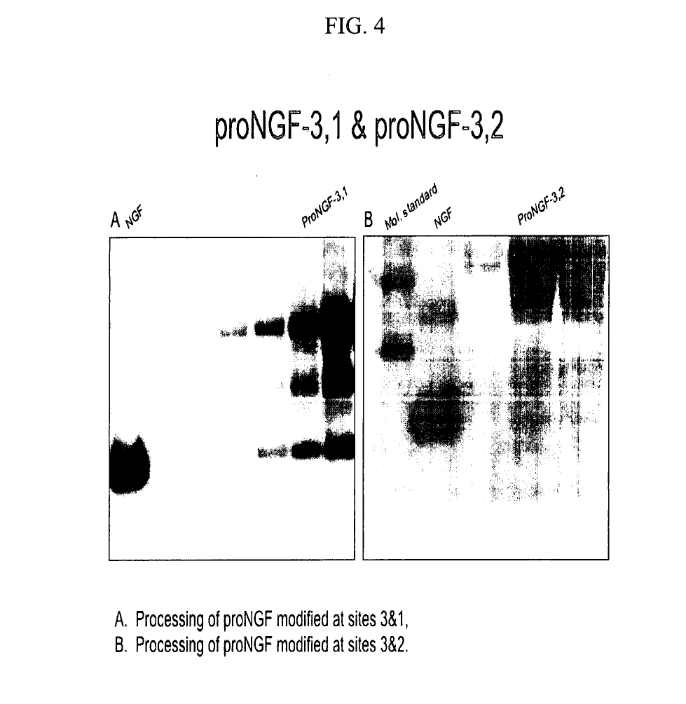

ProNGF with sites 1 and 2 mutated (proNGF-1,2) gave all NGF supporting the earlier finding that site 3 is the major site (FIG. 2). ProNGF with mutations at sites 3 and 1 (proNGF-3,1) gave proNGF, smaller percent of NGF, and one processing intermediate of about 22 kDa (FIG. 4A). ProNGF with sites mutated at 3 and 2 (proNGF-3,2) gave a pattern similar to proNGF mutated at sites 3 and 1 except with reduced amount of processing (FIG. 4B).

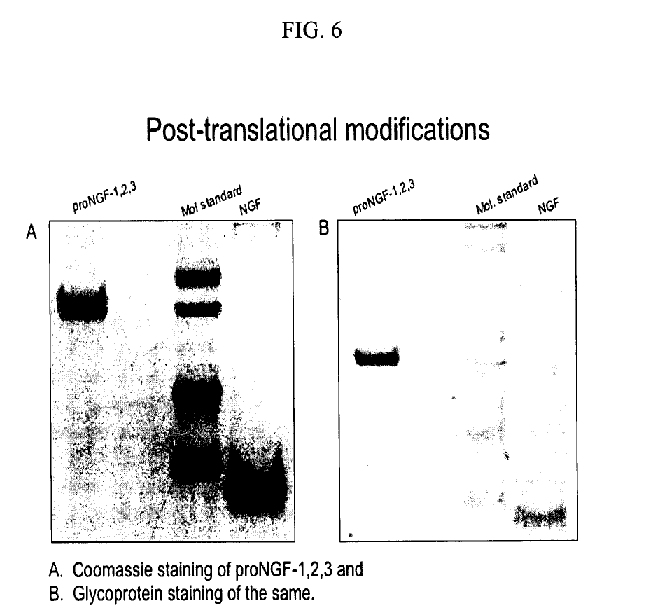

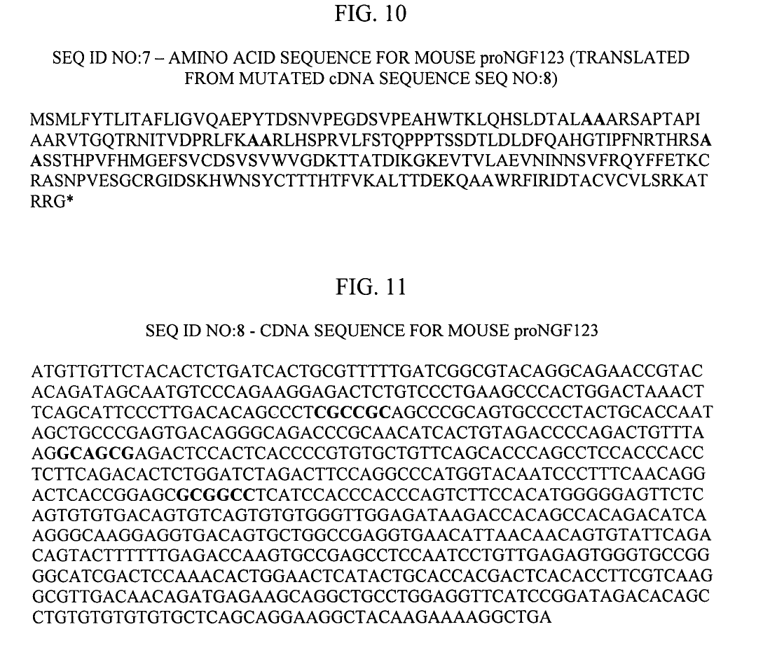

ProNGF with mutations at all the three processing sites (proNGF-1,2,3, also known as proNGF123) is expressed as essentially all proNGF in stably transfected Sf-21 insect cell line. Thus, mutating processing sites 1 and 2 in addition to site 3 significantly improved the stability of proNGF. Wild type proNGF, when made in bacteria, is produced as proNGF without any processing. Although it is not processed inside the bacteria, it is present in the inclusion bodies and upon refolding is still susceptible to proteolysis. Also, bacterial proNGF does not have post translational modifications like glycosylations. ProNGF123, made in stable insect cell line, is folded and glycosylated inside the eukaryotic cells and hence is closest to the native proNGF (FIG. 6). The amino acid sequence of the proNGF123 from mutated mouse β NGF cDNA is shown in FIG. 10 (SEQ ID NO:7), which is translated from the mutated cDNA SEQ ID NO:8 shown in FIG. 11. The mutated sites in SEQ ID NOS:7 and 8 are shown in bold.

### Example 6

### Production of Stable proNGF123

ProNGF123 is stably produced in Sf-21 insect cell line. These cells are grown in spinner flasks in expression medium for laboratory scale protein production. Half of the expression medium with protein is harvested every fourth day for purification and the volume of the rest is made up again to keep the production of proNGF123 continuous. Insect cells are completely healthy for at least 4 such harvests when the entire medium is harvested and new production run can be started. The stable insect cell line is therefore a good solution to production of large quantities of stable and biologically active proNGF for structural and cellular studies.

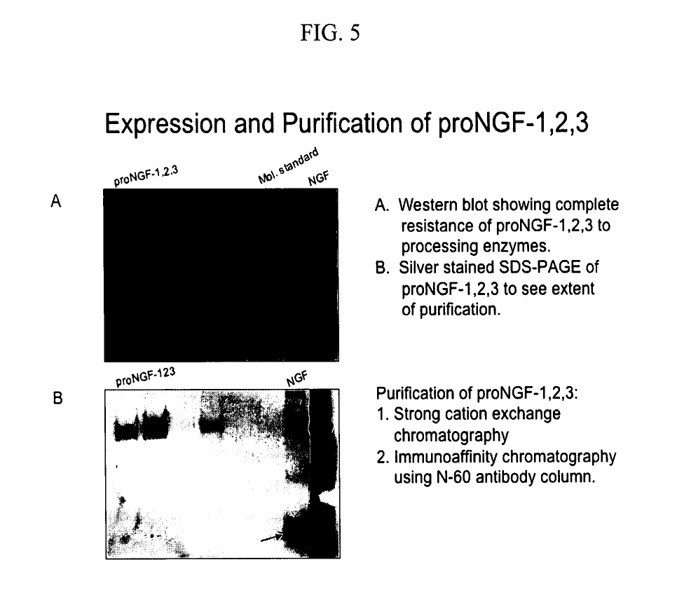

Purification of proNGF123 involved ion exchange chromatography and immunoaffinity chromatography. For ion exchange, strong cation exchanger SP fast flow column was used. This step purified and concentrated proNGF123 about 30 fold. This semi-purified proNGF123 was then purified to being about 99% pure and 300 fold concentrated using immunoaffinity column made with N-60 antibody (FIG. 5). N-60 antibody recognizes a conformational epitope on NGF. Purity of proNGF-123 purified using above mentioned two steps was checked with silver staining. Final yield of purified proNGF is about 1 mg/L of expression medium.

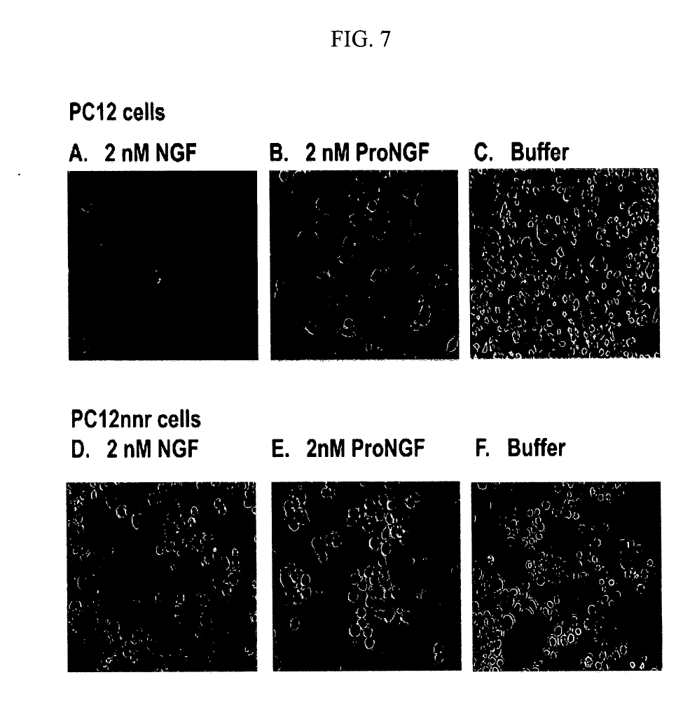

Biological activity of proNGF123 was tested on PC-12 cells which express TrkA receptor, p75NTR, and the Sortilin co-receptor. While mature NGF induces neurite outgrowth and stimulates survival in PC-12 cells at 2 nM concentration, proNGF123 induces cell death at 2 nM concentration. In the first 12 hours of proNGF123 treatment, PC-12 cells show some dendritic outgrowth, but don't generate neurites even after 120 hours of proNGF123 treatment (FIG. 7). Trypan blue analysis 72 hours post treatment with proNGF123 shows more than 90% cell death (FIG. 7). This result is in agreement with the hypothesis proposing induction of cell death by proNGF using p75NTR and sortilin receptor since PC-12 cells have both of these receptors on their cell membranes. Although this is an anticipated result, similar result has not been reported with the proNGF modified only at site 3 in PC-12 cells, probably because of potential processing of proNGF-3 by PC-12 cells or any processing enzymes in the growth medium of PC-12 cells. The proNGF123 bound to TrkA, as shown by surface plasmon resonance studies, but did not activate it, as shown by receptor phosphorylation experiment; in contrast, proNGF3 was capable of activating TrkA.

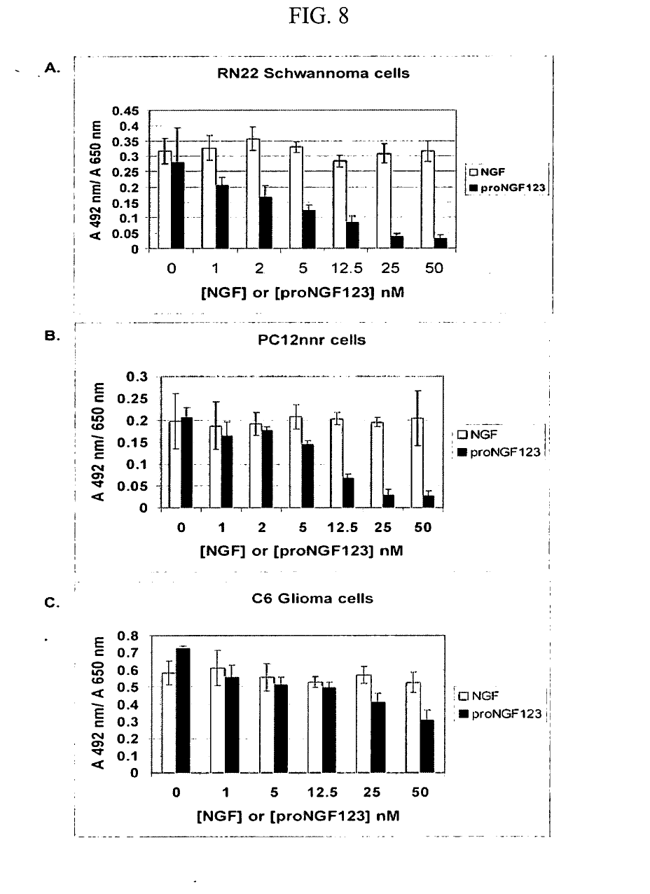

Survival for PC 12, PC 12nnr, C6 glioma, and RN22 schwannoma cells was quantitated by counting dead and live cells in trypan blue exclusion assay and/or XTT cell survival assay (FIG. 8). Concentrations of NGF and ProNGF123 used varied from 1 nm to 50 nM. The XTT survival assay confirms the results of trypan blue exclusion assay that proNGF123 induces cell death in various cell types which contain p75^{NTR}. Due to significantly lower metabolic rate of PC12 cells than the rest of the cell lines used and low number of cells plated, the XTT assay was not used to analyze PC12 cells. Instead, PC12 cell survival was quantitated by counting the live and dead cells in the trypan blue exclusion assay and confirmed that proNGF123 does not support survival.

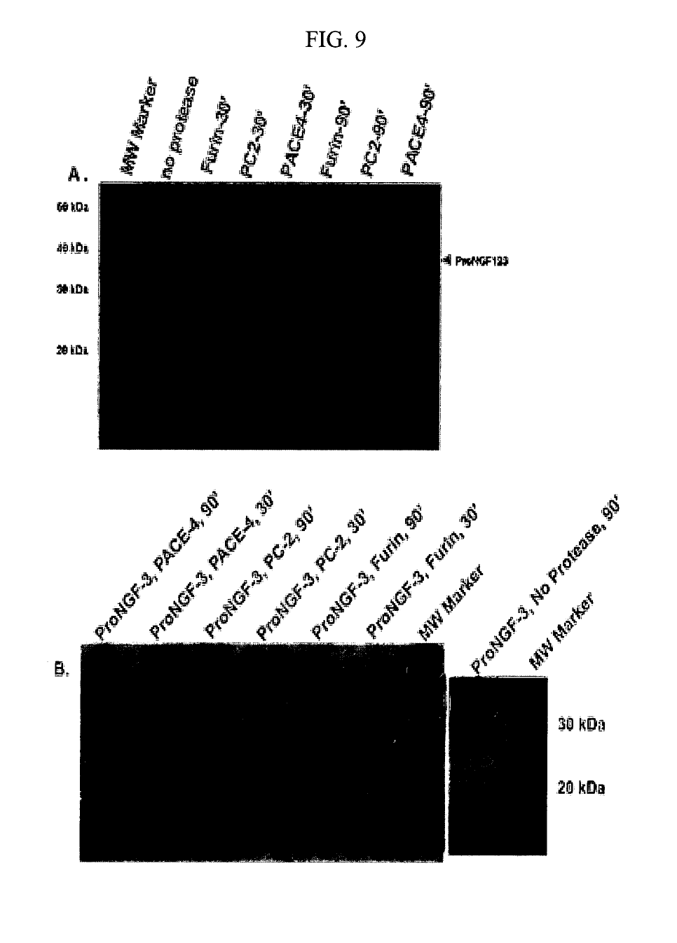

Convertases furin and PACE-4 have been shown to process proNGF to mature NGF (Seidah, N G, Benjannet, S, Pareek, S, Savaria, D, Hamelin, J, Goulet, B, Laliberte, J, Lazure, C, Chretien, M & Murphy, R A (1996) Cellular processing of the nerve growth factor precursor by the mammalian pro-protein convertases. Biochem J 314, 951-60). Stability of proNGF123 was tested by determining it's susceptibility to proteolysis by Furin, PACE-4, and PC-2 enzymes. PACE-4 and PC-2 enzymes were made using baculoviral expression system. Activities of purified PACE-4 and PC-2, relative to that of Furin (New England Biolabs, Ipswich, M A), were determined using the fluorogenic substrate peptide Boc-R-V-R-R-AMC and a TECAN plate reader. One microgram proNGF123 was exposed to either 2 or 4 units of each of these enzymes for 30 and 90 minutes duration at their optimal temperatures. Western blot analysis of the proteolysis experiment did not show any conversion of proNGF-123 to either mature NGF or any other intermediate with any of the three proteases thereby indicating complete resistance of proNGF123 to degradation by these enzymes (FIG. 9A). On the other hand, proNGF3, which was expressed as a mixture of proNGF, mature NGF, and intermediately processed forms, was stable to further processing by some proconvertases (FIG. 9B). Although furin proteolyzed full length proNGF3 to an intermediate form of about 26 kDa, neither furin nor any other proconvertase tested processed it to mature NGF significantly. The 26 kDa intermediate is consistent with processing of proNGF3 at site 1. This suggests that the stability conferred to the proNGF123 by additional mutations at sites 1 and 2 might be because of resistance to one of the other proconvertases known to be responsible for processing of neurotrophins like PC-1, PC-5, or PC-5/6B and not furin, PC-2 or PACE-4. ProNGF123 is not only more resistant to proteolysis than proNGF3 intracellularly, it is also more stable for storage as observed by effect of repeated freeze thaw cycles on each of these mutations.

While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein.

### REFERENCES

1. Baldwin, A N, Bitler, C M, Welcher, A A & Shooter, E M (1992) Studies on the structure and binding properties of the cysteine-rich domain of rat low affinity nerve growth factor receptor (p75NGFR). J. Biol. Chem. 267, 8352-8359

2. Barbacid, M (1994) The Trk family of neurotrophin receptors. J. Neurobiol. 25, 1386-1403

3. Bibel, M & Barde, Y A (2000) Neurotrophins: key regulators of cell fate and cell shape in the vertebrate nervous system. Genes Dev 14, 2919-37.

4. Bibel, M, Hoppe, E & Barde, Y A (1999) Biochemical and functional interactions between the neurotrophin receptors trk and p75NTR. EMBO J 18, 616-22

5. Eide, F F, Lowenstein, D H & Reichardt, L F (1993) Neurotrophins and their receptors—current concepts and implications for neurologic disease. Exp. Neurol. 121, 200-214

6. Friedman, W J (2000) Neurotrophins induce death of hippocampal neurons via the p75 receptor. J Neurosci 20, 6340-6.

7. Friedman, W J & Greene, L A (1999) Neurotrophin signaling via Trks and p75. Exp Cell Res 253, 131-42

8. Harrington, A W, Leiner, B, Blechschmitt, C, Arevalo, J C, Lee, R, Morl, K, Meyer, M, Hempstead, B L, Yoon, S O & Giehl, K M (2004) Secreted proNGF is a pathophysiological death-inducing ligand after adult CNS injury. Proc Natl Acad Sci USA 101, 6226-30. Epub Mar. 16, 2004.

9. Heymach Jr., J V, Kruttgen, A, Suter, U, Shooter, E M (1996) The Regulated Secretion and Vectorial Targeting of Neurotrophins in Neuroendocrine and Epithelial Cells. JBC 271, 25430-25437.

10. Kliemannel, M, Rattenholl, A, Golbik, R, Balbach, J, Lilie, H, Rudolph, R & Schwarz, E (2004) The mature part of proNGF induces the structure of its pro-peptide. FEBS Lett 566, 207-12.

11. Kong, H, Kim, A H, Orlinick, J R & Chao, M V (1999) A comparison of the cytoplasmic domains of the Fas receptor and the p75 neurotrophin receptor. Cell Death Differ. 6, 1133-42.

12. Lee, R, Kermani, P, Teng, K K & Hempstead, B L (2001) Regulation of cell survival by secreted proneurotrophins. Science 294, 1945-8.

13. Levi-Montalcini, R (1987) The nerve growth factor 35 years later. Science 237, 1154-1162

14. Maness, L M, Kastin, A J, Weber, J T, Banks, W A, Beckman, B S & Zadina, J E (1994) The neurotrophins and their receptors: structure, function, and neuropathology. Neurosci. Biobehav. Rev. 18, 143-159

15. McDonald, N Q & Hendrickson, W A (1993) A structural superfamily of growth factors containing a cystine knot motif. Cell 73, 421-424

16. McInnes, C & Sykes, B D (1997) Growth factor receptors: structure, mechanism, and drug discovery. Biopolymers. 43, 339-66.

17. Mufson, E J, Lavine, N, Jaffar, S, Kordower, J H, Quirion, R & Saragovi, H U (1997) Reduction in p140-TrkA receptor protein within the nucleus basalis and cortex in Alzheimer's disease. Exp Neurol 146, 91-103

18. Nykjaer, A, Lee, R, Teng, K K, Jansen, P, Madsen, P, Nielsen, M S, Jacobsen, C, Kliemannel, M, Schwarz, E, Willnow, T E, Hempstead, B L & Petersen, C M (2004) Sortilin is essential for proNGF-induced neuronal cell death. Nature 427, 843-8.

19. Rattenholl, A, Lilie, H, Grossmann, A, Stern, A, Schwarz, E & Rudolph, R (2001) The pro-sequence facilitates folding of human nerve growth factor from Escherichia coli inclusion bodies. Eur J Biochem 268, 3296-303.

20. Seidah, N G, Benjannet, S, Pareek, S, Savaria, D, Hamelin, J, Goulet, B, Laliberte, J, Lazure, C, Chretien, M & Murphy, R A (1996) Cellular processing of the nerve growth factor precursor by the mammalian pro-protein convertases. Biochem J 314, 951-60

21. Server, A C & Shooter, E M (1977) Nerve growth factor. Adv. Protein. Chem. 31, 339-409

22. Wiesmann, C & de Vos, A M (2001) Nerve growth factor: structure and function. Cell Mol Life Sci 58, 748-59.

23. Wiesmann, C, Ultsch, M H, Bass, S H & de Vos, A M (1999) Crystal structure of nerve growth factor in complex with the ligand-binding domain of the TrkA receptor. Nature 401, 184-188.

We claim:

1. A mutated mammalian stable pro nerve growth factor (proNGF) derived from a wild type mammalian proNGF, the wild type proNGF having a pro domain and a mature domain, a first dibasic protease sensitive site with contiguous basic amino acids in the pro domain, a second dibasic protease sensitive site with contiguous basic amino acids in the pro domain, and a third dibasic protease sensitive site with contiguous basic amino acids in the pro domain, wherein each of the basic amino acid residue in each of the dibasic site in the wild type proNGF is replaced by a non-basic amino acid.

2. The proNGF of claim 1, wherein the non-basic amino acid is neutral or acidic.

3. The proNGF of claim 2 wherein the neutral amino acid is alanine.

4. The proNGF of claim 1, wherein the proNGF is a human proNGF.

5. A method for making a mutated stable mammalian pro nerve growth factor (proNGF) from a wild type proNGF, the wild type proNGF having a pro domain and a mature domain, a first dibasic protease sensitive site having contiguous amino acids in the pro domain, a second dibasic protease sensitive site having contiguous amino acids in the pro domain, and a third dibasic protease sensitive site having contiguous amino acids in the pro domain, the method comprising:
(a) providing a cDNA for the wild type proNGF;
(b) subcloning the cDNA into a cell vector;
(c) mutating the cDNA at sites corresponding to each of the dibasic sites to non-basic amino acids to obtain a plasmid with mutated cDNA in the cell vector;
(d) transfecting the plasmid or the cell vector containing the plasmid into a host cell to establish a stably transfected cell line which constituitively expresses the cDNA, and
(e) culturing the host cell to allow expression of the mutated proNGF.

6. The method of claim 5, further comprising purifying the mutated proNGF from the cell culture in step (e).

7. The method of claim 5, wherein the non-basic amino acid is acidic or neutral.

8. The method of claim 7, wherein the neutral amino acid is alanine.

9. The method of claim 5, wherein the proNGF is a human proNGF.

10. The method of claim 5, wherein a native stop codon in the cDNA is induced to avoid 6-histidine residue (6 His) tag (SEQ ID NO: 12) in the vector.

11. The method of claim 5, wherein the cell is selected from the group consisting of mammalian cells, avian cells, insect cells, plant cells, and yeast cells.

12. The method of claim 5, wherein the cell is an insect cell.

13. The method of claim 12, wherein the insect cell is a Sf-21 cell.

14. The method of claim 12 wherein the insect cell is a Sf-9 cell.

15. The method of claim 12 wherein the insect cell is a Hi-5 cell.

16. The method of claim 5, wherein the cell vector is a pIZT/his/VR vector.

17. The method of claim 5, wherein the cell vector is a pBlueBac vector.

15. A stable mutated pro nerve growth factor (proNGF) comprising SEQ ID NO:7 or SEQ ID NO:11.

16. A cDNA construct for a mutated pro nerve growth factor (proNGF), wherein the cDNA construct translates into the mutated proNGF of claim 1.

17. The cDNA construct of claim 16 comprising SEQ ID NO:8.
